(19) United States
(12) Patent Application Publication
(10) Pub. No.: US 2025/0010029 A1
(43) Pub. Date: Jan. 9, 2025

(54) MEDICAL ACCESS CANNULAS AND ASSOCIATED METHODS

(71) Applicant: Boston Scientific Scimed, Inc., Maple Grove, MN (US)

(72) Inventors: Joseph MAYO, Medford, MA (US); Ramin N. TEHRANI, Jamaica Plain, MA (US); Lisa Margaret SANGREE, Boston, MA (US); David CALLAGHAN, Ashland, MA (US)

(73) Assignee: Boston Scientific Scimed, Inc., Maple Grove, MN (US)

(21) Appl. No.: 18/761,439

(22) Filed: Jul. 2, 2024

Related U.S. Application Data
(60) Provisional application No. 63/511,900, filed on Jul. 5, 2023.

(51) Int. Cl. A61M 25/00

(52) U.S. Cl. CPC A61M 25/0023

(57) ABSTRACT

A medical device may comprise a tube including: a proximal portion; and a distal portion having a slotted section with a plurality of slots formed therein. A proximalmost slot of the plurality of slots may have a first length in a circumferential direction about the tube. A distalmost slot of the plurality of slots may have a second length in the circumferential direction about the tube. The first length may be smaller than the second length. The medical device may further comprise a hub coupled to a proximal portion of the tube. Rotation of the hub may be configured to rotate the proximal portion of the tube about a longitudinal axis of the proximal portion of the tube.

### CROSS-REFERENCE TO RELATED APPLICATIONS

This application claims the benefit of priority under 35 U.S.C. § 119 from U.S. Provisional Application No. 63/511,900, filed Jul. 5, 2023, which is incorporated by reference herein in its entirety.

### TECHNICAL FIELD

This disclosure relates generally to medical devices and, more particularly, to medical access cannulas.

### BACKGROUND

Elongated medical devices, such as guidewires and cannulas, may be used to provide access into a vessel, channel, lumen, or other chamber of a subject. The guidewire or cannula, once placed, may provide an access path for subsequent therapeutic or diagnostic devices used in the procedure. Manipulating the end of the guidewire or cannula to provide proper direction to such devices can be difficult and can result in damage to nearby tissue. There is a need, therefore, for improved access devices that provide proper directionality to the subsequent therapeutic and diagnostic devices.

### SUMMARY OF THE DISCLOSURE

Each of the aspects disclosed herein may include one or more of the features described in connection with any of the other disclosed aspects. Aspects of the disclosure relate to, among other things, systems, devices, and methods for medical device cannulas.

According to an example, a medical device may comprise a tube including: a proximal portion; and a distal portion having a slotted section with a plurality of slots formed therein. A proximalmost slot of the plurality of slots may have a first length in a circumferential direction about the tube. A distalmost slot of the plurality of slots may have a second length in the circumferential direction about the tube. The first length may be smaller than the second length. The medical device may further comprise a hub coupled to a proximal portion of the tube. Rotation of the hub is configured to rotate the proximal portion of the tube about a longitudinal axis of the proximal portion of the tube.

Any of the medical devices disclosed herein may have any of the following features, alone or in any combination. Each slot of the plurality of slots may extend approximately perpendicularly to a longitudinal axis the tube. The slotted section may include a longitudinally extending unslotted spine. The spine may be a first spine, and the slotted section may include a second longitudinally extending unslotted spine. Each slot of the plurality of slots may extend between the first spine and the second spine. The tube may include an array of slots having a first, proximal portion of slots and a second, distal portion. Each slot of the plurality of slots of the second portion may have approximately a same circumferential length. Lengths of the plurality of slots in the first portion may taper from a first circumferential length at a proximal end of the first portion to a second circumferential length at a distal end of the first portion. The second circumferential length may be greater than the first circumferential length. In a relaxed state, the distal portion may include a curved portion having a curved central longitudinal axis. In the relaxed state, the tube may have an approximately “J” shape. A jacket may cover at least a portion of the tube. The jacket may extend distally beyond a distalmost end of the tube such that the jacket forms a distal tip of the medical device. The distal tip may taper radially inward toward a distal end of the distal tip. The medical device may further comprise an electrosurgical component that is configured to advance over the tube. The distal portion may further include an unslotted section distal to the slotted section.

In a further example, a medical device may comprise: a tube including: a proximal portion; and a distal portion having a slotted section. The slotted section may include a longitudinally extending unslotted spine and a plurality of slots extending from the unslotted spine. In a relaxed state, the distal portion may include a curved portion having a curved central longitudinal axis. The medical device may further comprise a hub coupled to a proximal portion of the tube.

Any of the devices disclosed herein may include any of the following features, alone or in any combinations. The spine may be a first spine. The slotted section may include a second longitudinally extending unslotted spine. Each slot of the plurality of slots may extend between the first spine and the second spine.

In a further example, a medical device may comprise: a tube including: a proximal portion; and a distal portion having an array of slots having a first, proximal portion of slots and a second, distal portion of slots. Each slot of the second portion may have approximately a same circumferential length. Lengths of the slots in the first portion may taper from a first circumferential length at a proximal end of the first portion to a second circumferential length at a distal end of the first portion. The second circumferential length may be greater than the first circumferential length. The medical device may further include a hub coupled to a proximal portion of the tube.

Any of the devices disclosed herein may include any of the following features, alone or in any combination. In a relaxed state, the distal portion may include a curved portion having a curved central longitudinal axis.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1 is a side view of a medical device, according to an embodiment.

FIG. 2 is a side view of a portion of a shaft of the medical device of FIG. 1, according to an embodiment.

FIGS. 3A-3B depict a tube of the shaft of FIG. 2.

FIG. 4 depicts the tube of FIGS. 3A-3B if it were cut and unrolled.

FIG. 5 is a schematic drawing of aspects of the tube of FIGS. 3A-3B.

FIG. 6 is side view of the portion of the shaft of FIG. 2 with an accessory device inserted therethrough.

FIG. 7 is a side view of a portion of an alternative tube for use with the medical device of FIG. 1.

FIG. 8 depicts a portion of the medical device of FIG. 1 with an electrosurgical device.

FIG. 9 depicts an exemplary medical method using the device of FIG. 1.

### DETAILED DESCRIPTION

In aspects of the disclosure, a medical device, such as an access cannula, may have features that facilitate advancing a guidewire through the medical device and manipulating a position of the guidewire using the medical device. In embodiments, the medical device may include a flexible distal portion that has a resting approximately “J” shape. Slots in the distal portion of the medical device may result in flexibility of the medical device, and the resting “J” shape may be formed by, for example, heat-setting or memory forming the distal portion of the medical device. A stylet (e.g., a sharp stylet) may be advanced through the medical device to straighten the medical device and to puncture through tissue. A guidewire may be advanced through the medical device while it has a resting “J” shape, and the “J” shaped medical device may be rotated via a handle to manipulate the guidewire. The handle of the medical device may include interfaces for sources of fluids and/or for aspiration, which may be transmitted and/or applied via the medical device. The disclosed aspects may help to allow for smooth puncture of tissue, as well as atraumatic guidewire manipulation. Furthermore, the medical device may help to provide for electrical insulation and thermal stability. Aspects of the medical device, such as the slot pattern at the distal portion of the medical device, may help to provide for increased echogenic visualization of the medical device.

The devices disclosed herein may be utilized for medical procedures, such as endoscopic ultrasound (“EUS”) procedures. In some examples, EUS procedures may be performed to access structures of a pancreatico-biliary of a subject such as, for example, when endoscopic retrograde cholangiopancreatography (ERCP) procedures cannot be successfully performed. In aspects, EUS drainage procedures may be performed under EUS guidance in order to direct a guidewire out of a papilla of Vater of a subject to perform a rendezvous procedure or in order to directly stent a dilated structure into a subject's duodenum or stomach. Access to a target site may be achieved via trans-gastric or trans-duodenal puncture into the target site. For example, using the disclosed devices, a user may pass a stylet through the medical device in order to puncture into the target site and thereby access the target site. Access may confirmed via fluoroscopic contrast injection and/or aspiration of bile/pancreatic fluids through the medical device. Additionally or alternatively, saline, sterile water, or another fluid may be injected via the medical device, for example, to help provide lubrication for a guidewire. After accessing the target site, a guidewire may be passed through the medical device to, for example, advance through the papilla for a rendezvous procedure or to secure access for trans-mural stenting. The guidewire may be directed by rotating the medical device with the medical device in a “J” shape. Because the medical device may have an atraumatic tip, the manipulation of the guidewire may be achieved without damaging nearby tissue or shearing the guidewire. Thereafter, a desired procedure may be performed using the guidewire.

Both the foregoing general description and the following detailed description are exemplary and explanatory only and are not restrictive of the invention, as claimed. As used herein, the terms “comprises,” “comprising,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “diameter” may refer to a width where an element is not circular. The term “circumference” may refer to a perimeter when an element is not circular. The term “distal” refers to a direction away from an operator, and the term “proximal” refers to a direction toward an operator. The term “exemplary” is used in the sense of “example,” rather than “ideal.” The term “approximately,” or like terms (e.g., “substantially”), includes values +/−10% of a stated value.

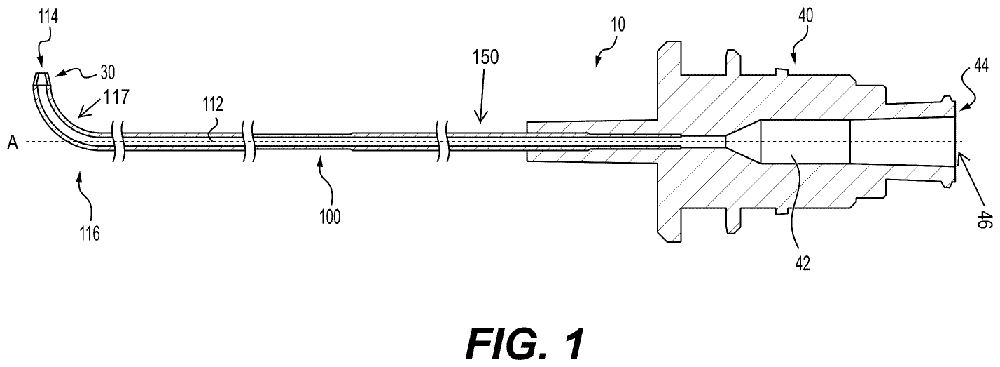

Referring to FIG. 1, a medical device 10 according to an embodiment is shown. FIG. 1 shows a cross-sectional view of medical device 10. Medical device 10 may be or include an access cannula, which may provide access to a stylet for puncturing tissue, allow passage of fluids and/or suction, and direct/steer a guidewire passed therethrough. Medical device 10 may include a shaft 100, including a distal portion 116 terminating in a distal tip 30 at a distalmost end of shaft 100. Shaft 100 have a lumen 112 extending therethrough, along a longitudinal axis of shaft 100. Lumen 112 may terminate distally in a distal opening 114 in distal tip 30.

A hub 40 may be coupled to a proximal end of shaft 100. Hub 40 may be similar to hubs that are used with manual fine needle aspiration biopsy needles and may have any of the properties thereof. Hub 40 may be a component of a handle (e.g., incorporated into a distal end of a handle). For example, hub 40 may be incorporated into a handle having any of the features of handles described in U.S. patent application Ser. No. 17/137,070, published as U.S. Patent Application Publication No. 2021/0205012, which is incorporated by reference herein in its entirety. For example, the handle may have automatic locking features and may facilitate movement of shaft 100. For example, hub 40 may have any of the properties of the rotation assembly of U.S. patent application Ser. No. 17/137,070. Hub 40 may include a lumen 42 extending longitudinally therethrough. Lumen 42 of hub 40 may be in fluid communication with lumen 112 of shaft 100. A proximal end of lumen 42 may include a fitting 44, such as a Luer fitting or other type of fitting. Lumen 42 may be open on a proximal end thereof at a proximal opening 46, such that a syringe or other device attached to fitting 44 may be able to introduce fluid into lumen 42 (and, therefore, lumen 112) via opening 46 and/or apply negative pressure/suction. As discussed below, devices such as stylets or guidewires also may be passed into opening 46, through lumen 42, into lumen 112, and out of distal opening 114.

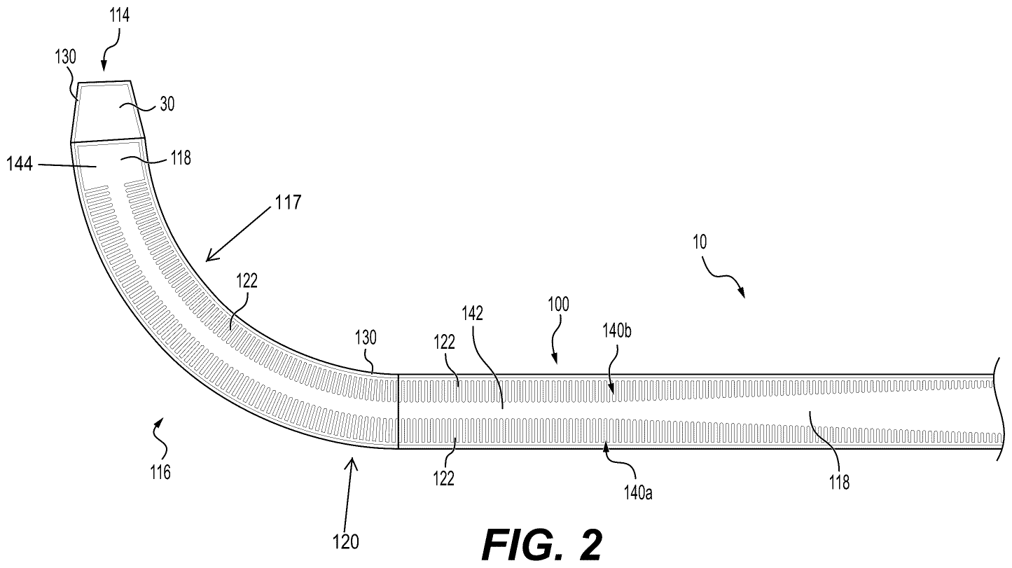

FIG. 2 shows details of a portion of shaft 100 of medical device 10. Shaft 100 may include a tube 118. At least a distal portion 116 of shaft 100 may also include a jacket 130, discussed in further detail below. Jacket 130 is depicted as being transparent in FIG. 2 in order to depict features of tube 118; however, jacket 130 may have any suitable color, opacity, etc., Although jacket 130 is described herein as being disposed on distal portion 116, it will be appreciated that jacket 130 may extend along one or more additional portions of shaft 100 (e.g., along an entirety of shaft 100). Lumen 112 (not labeled in FIG. 2) may extend through tube 118 and through a portion of jacket 130 (e.g., distal tip 30) that extends distally of tube 118. Further details of jacket 130 and distal tip 30 are provided below.

Tube 118 may be formed from Nitinol or another material that may be visualized under X-ray (e.g., fluoroscopic visualization). Alternatively, tube 118 may be formed from another material, and other portions of device 10 (e.g., jacket 130) may be visualized on X-ray. A thickness of a wall of tube 118 and a geometry of tube 118 may be such that tube 118 is visible on X-ray, even without a stylet (discussed in further detail below) or guidewire inserted through lumen 112. Tube 118 may extend from hub 40 (FIG. 1) to a proximal end of distal tip 30 (distal tip 30 is discussed in further detail below). Alternatively, tube 118 may extend along only a portion of shaft 100 and/or may itself form distal tip 30.

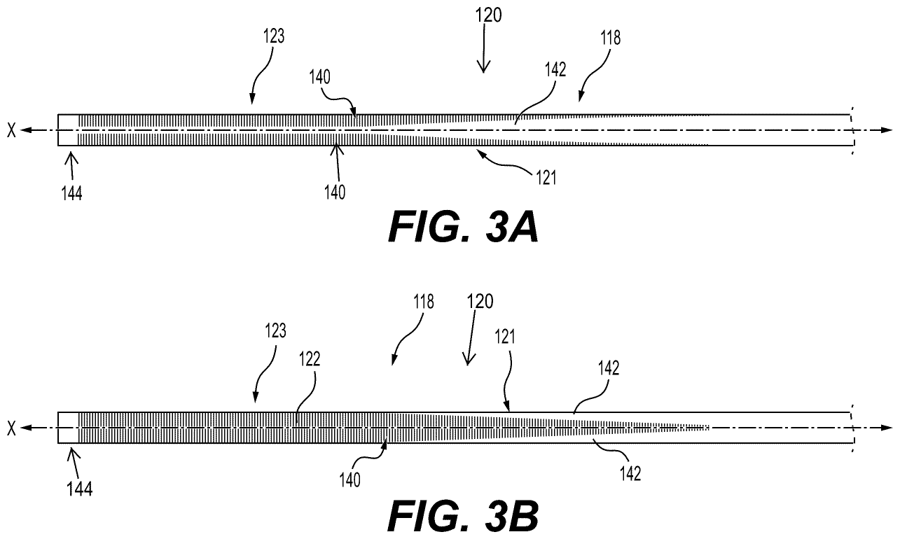

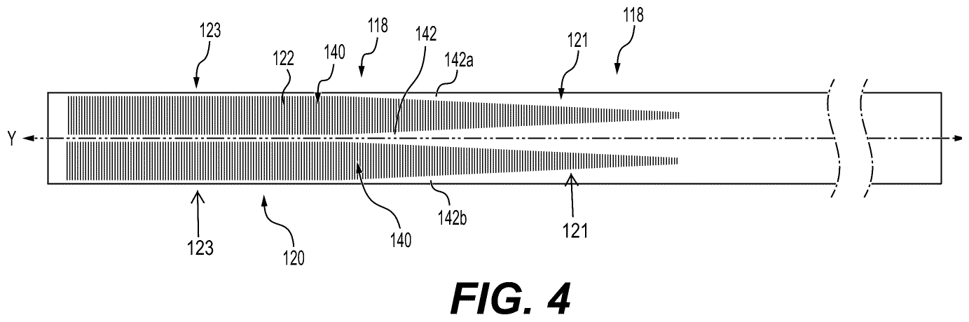

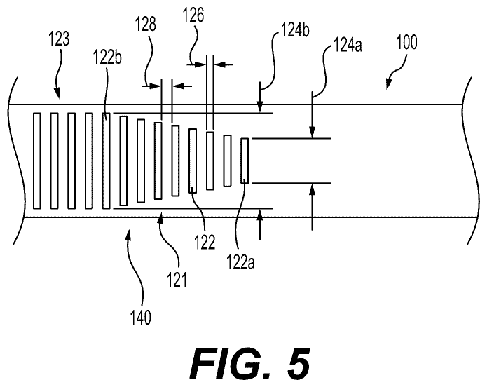

Tube 118 (e.g., a distal portion of tube 118) may include a slotted section 120. Proximal portions of tube 118 (e.g., portions of tube 118 in a proximal portion 150 of shaft 100, shown in FIG. 1) may be unslotted. Furthermore, a distalmost portion 144 of tube 118 may be unslotted. Slotted section 120 may include slots 122 extending through an entire thickness of a wall of tube 118. Slots 122 may be formed by, for example, laser cutting portions of tube 118. In some embodiments, slotted section 120 may be positioned along a portion or an entirety of distal portion 116 of shaft 100. FIGS. 3A-3B show a distal portion of tube 118 without jacket 130 and distal tip 30 to depict further details of slotted section 120. FIG. 3B shows tube 118 rotated 90 degrees about a longitudinal axis X of tube 118 with respect to the configurations of FIGS. 2 and 3A. FIG. 4 shows tube 118 if it were cut parallel to longitudinal axis X and flattened to form a sheet. FIG. 4 is merely illustrative to show features of a pattern of slots 122. FIG. 5 is a schematic, not-to-scale image showing a portion of tube 118 to illustrate measurements associated with slots 122.

As shown by considering FIGS. 3A-3B and 4 together, tube 118 may include two arrays 140 of slots 122 separated by two spines 142. Spines 142 may be portions of tube 118 that extend longitudinally along tube 118 (approximately parallel to longitudinal axis X of tube 118) and do not have slots 122 formed therein (i.e., are unslotted). As shown in FIG. 4, for illustrative purposes, tube 118 has been “cut” so that one of spines 142 is divided into two pieces 142a, 142b. However, in medical device 10, pieces 142a, 142b would not be separate pieces and would be a single spine 142. Pieces 142a, 142b are merely labeled in FIG. 4 for ease of illustration of the elements of tube 118. Overall shapes of arrays 140 may be symmetrical to one another about a plane Y that extends along the line shown in FIG. 4 of the “unrolled”/“flattened” tube 118 and into and out of the page of FIG. 4 (i.e., perpendicular to the plane of the page). In other words, arrays 140 may be symmetrical to one another about a plane that extends along central longitudinal axis X (FIGS. 3A-3B) of tube 118 and through a center of each spine 142.

Slots 122 may extend partially around a circumference of tube 118. A circumferential direction may refer to a direction around a circumference of tube 118, perpendicular to a longitudinal axis X of tube 118 at a particular direction (e.g., at a location of each of slots 122). Each array 140 may include a first portion 121 and a second portion 123. First portion 121 may be proximal of second portion 123. Slots 122 in second portion 123 of array 140 may have approximately a uniform/same length extending around a portion of a circumference of tube 118. For example, slots 122 may extend slightly less than halfway around a circumference of tube 118. Slots 122 in first portion 121 of array 140 may have varying lengths in a circumferential direction around tube 118. As shown in FIGS. 3A-4, lengths of slots 122 along a circumferential direction of tube 118 may decrease moving in a proximal direction along first portion 121 of array 140. Thus, first portion 121 of array 140 may have a tapered shape, such that circumferential lengths of slots 122 decrease in a proximal direction, and a circumferential length (a length in a direction of a circumference of tube 118) of spines 142 increases along the proximal direction. More proximal slots 122 of first portion 121 may have a smaller circumferential length than more distal slots 122 of first portion 121.

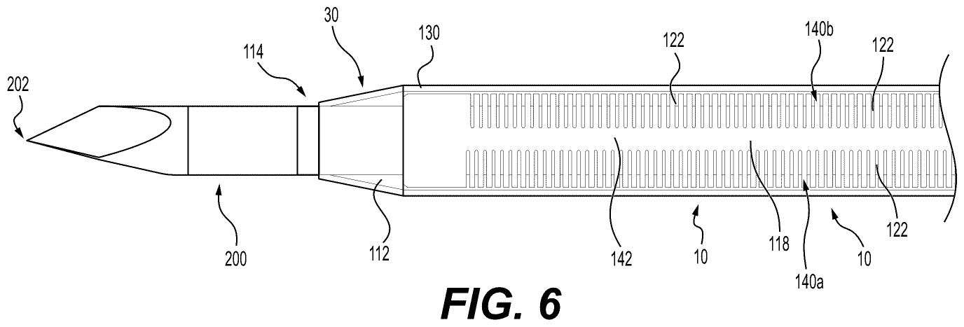

As shown in FIG. 2, slots 122 of a first array 140a of arrays 140 may alternate with slots of a second array 140b of arrays 140 across spine 142. In other words, a line extending circumferentially around tube 118 may intersect a slot 122 from only one of first array 140a and second array 140b. Rephrased another way, across spine 142 from one slot 122 of first array 140a may be a space between slots 122 of second array 140b. Of note, the slots 122 of FIG. 2 are not necessarily drawn to scale, and spaces between slots 122 may be wider in an axial/longitudinal direction than slots 122 (as shown in FIG. 6, which is described in further detail below).

With reference to FIG. 5, first portion 121 of array 140 may have slots 122 with a circumferential length about tube 118 that gradually increases along a distal direction from a first length 124a to a second length 124b. A first slot 122a having the first length 124a may be the proximalmost slot of first portion 121. As depicted, a second slot 122b having second length 124a may be a distalmost slot of first portion 121. The slots 122 disposed between the first slot 122a and the second slot 122b may have varying circumferential lengths between 124a and 124b such that first portion 121 of array 140 may have a tapered shape. As depicted, each of the slots 122 of the first portion 121 may have a width 126 in an axial/longitudinal direction that is the same or substantially the same as each of the other slots 122. In other embodiments, the width 126 may vary among the slots 122. Each of the slots 122 may be spaced apart from an adjacent slot by an axial distance 128. Portions of tube 118 between slots 122 may form an approximately ring shape when including portions of tube 118 that extend across spines 142. In some embodiments, axial distances 128 may be the same or substantially the same between slots 122, as depicted. In other embodiments, axial distances 128 may be varied. Axial distances 128 may be greater than or equal to the widths 126. This may help to ensure that shaft 100 maintains sufficient strength to prevent or decrease the occurrences of breakage or wear during operation of shaft 100 and/or to facilitate rotation of shaft 100.

Slots 122 of second portion 123 of array 140 may have an approximately uniform circumferential length. In some embodiments, a circumferential length of slots 122 of second portion 123 may be approximately the same as the second length 124b of first portion 121 (e.g., the length of distalmost slot 122b). In other embodiments, the lengths may differ. Axial widths of slots 122 of second portion 123 may be approximately equal to axial widths 126, and axial distances between slots 122 of second portion 123 may be approximately equal to axial distances 128.

The magnitude of the circumferential lengths 124a, 124b of slots 122, the widths 126 of slots 122, and the axial distance 128 between slots 122 may be particularized for the size and/or geometry of shaft 100. In some examples, length 124b may be approximately 0.02 inches to approximately 0.08 inches, approximately 0.04 inches to approximately 0.07 inches, or approximately 0.0567 inches. In examples, length 124a may be approximately 0.005 inches to approximately 0.011 inches, approximately 0.007 inches to approximately 0.010 inches, or approximately 0.0086 inches. In some examples, axial width 126 may be approximately 0.0007 inches to approximately 0.0013 inches, approximately 0.0008 inches to approximately 0.0012 inches, or approximately 0.0010 inches. Axial distance 128 may be approximately 0.0027 inches to approximately 0.0033 inches, approximately 0.0028 inches to approximately 0.0032 inches, or approximately 0.0030 inches. In examples, first portion 121 may have an axial length of approximately 0.30 inches to approximately 0.70 inches, approximately 0.40 inches to approximately 0.80 inches, or approximately 0.505 inches. A distance from a proximalmost end of second portion 123 to a distalmost end of tube 118 may be approximately 0.20 inches to approximately 0.60 inches, approximately 0.30 inches to approximately 0.55 inches, or approximately 0.444 inches. A distance from a distalmost end of second portion 123 to a distalmost end of tube 118 (i.e., an axial width of distalmost portion 144) may be approximately 0.010 inches to approximately 0.050 inches, approximately 0.020 inches to approximately 0.040 inches, or approximately 0.030 inches. A total axial length of tube 118 may be approximately 50.0 inches to approximately 90.0 inches, approximately 40.0 inches to approximately 80 inches, or approximately 68 inches (e.g., approximately 68.036 inches).

Returning to FIG. 2, jacket 130 may disposed around at least a portion of tube 118 (e.g., at least around a portion of tube 118 including slotted section 120). Jacket 130 may extend around an entire circumference of the portion of tube 118 about which jacket 130 is disposed. In some examples, jacket 130 may not extend around proximal portion 150 (FIG. 1) of shaft 100. Jacket 130 may be disposed radially outward of tube 118. In alternatives, jacket 130 may be disposed radially inward of tube 118. Jacket 130 may be formed of, for example, a polymer, such as a thermoplastic copolyester (TPC).

Jacket 130 may extend distally of a distalmost end of tube 118 to form distal tip 30. As shown in FIG. 2, jacket 130 may taper radially inwardly distally of tube 118 to opening 114. In alternatives, jacket 130 may be a tipped polymer jacket, with distal tip 30 rounded via machining, melting and reflowing within a tool, or heating to soften followed by reshaping. In alternatives, jacket 130 may include polytetrafluoroethylene (PTFE), Fluoroethylenepropylene (FEP), or thermoplastic polyurethanes (TPUs) such as Tecothane® or Pellethane®. Jacket 130 may further serve to seal lumen 112 (extending through tube 118) from an environment external to shaft 100, preventing ingress and egress of fluids through slots 122. Further features of distal tip 30 are discussed below, with respect to FIG. 6.

Distal portion 116 of shaft 100 may be flexible due to slots 122 and a flexibility of jacket 130. An arrangement of slots 122 may be such that shaft 100 is flexible in only one plane. Shaft 100 may be bendable in directions that are approximately perpendicular to a plane extending through centers of spines 142. For example, as shown in FIG. 2, shaft 100 may be flexible in an up/down direction of the page but not an in/out direction of the page. Slots 122 may decrease a stiffness of distal portion 116 of shaft 100 at the slotted section 120, which may enable such bending. In particular, the stiffness at the slotted section 120 may decrease in a distal direction as slots 122 become longer. By tapering the circumferential length of slots 122 from a first length 124a to a second length 124b that is larger than the first length 124a, the bending stress acting on shaft 100 while configured in a bent or J-shaped orientation may be gradual. The tapering of the circumferential length of slots 122 may reduce or avoid locations of concentrated stress. In other words, the tapered shape of arrays 140 may provide strain relief.

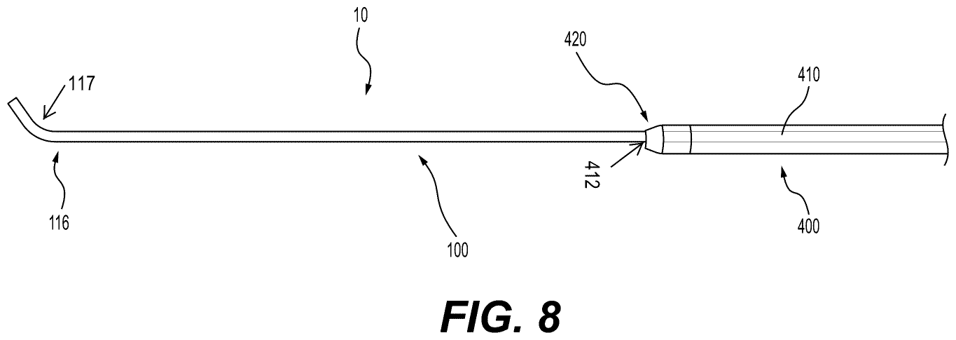

Shaft 100 may be biased into a “J” shape, as shown in FIGS. 1 and 2, such that shaft 100 has a “J-tip.” In other words, a bent portion 117 of shaft 100 may bend such that distal tip 30 faces a direction that is approximately perpendicular to a line A extending through a proximal portion 150 of shaft 100 and through hub 40 (and any unbent portion of distal portion 116), as shown in FIG. 1. Line A may be coaxial with a longitudinal axis of proximal portion 150 and hub 40, but may not be coaxial with a longitudinal axis of bent portion 117. A central longitudinal axis of shaft 100 may extend through a center of shaft 100 and may follow a curved path at bent portion 117. Thus, a longitudinal axis of bent portion 117 may have an angle with respect to (i.e., be transverse to) a longitudinal axis of proximal portion 150. A “longitudinal” direction may refer to a direction coaxial to or approximately parallel to the central longitudinal axis. Described still another way, bent portion 117 of shaft 100 may bend approximately 90 degrees. Such a bend is merely exemplary; as shown in FIG. 8, alternative bend shapes may be used.

The resting “J-tip” shape of shaft 100 may be produced by heat setting (or memory forming) bent portion 117 of shaft 100. For example, the “J-tip” shape of bent portion 117 may be held by a fixture or a curved mandrel and then bent portion 117 may be submerged in hot fluidized sand. Once cooled via quenching, a resting/biased shape of bent portion 117 of shaft 100 may be a “J” shape. As shown in FIG. 2, only a portion of slotted section 120 may be bent into the “J” shape at rest. Proximal portions of slotted section 120 (including, e.g., first portion 121) may be straight in a relaxed (resting) configuration.

In the relaxed, “J-tip” configuration of shaft 100, a guidewire or other flexible accessory device may be inserted into lumen 112 (e.g., via proximal opening 46 of hub 40, shown in FIG. 1). The guidewire may be extended distally out of distal opening 114. Because a proximal portion of tube 118 is rigid (does not include slots 122) and because distal portion 116 includes spines 142 between arrays 140 of slots 122, shaft 100 may be sufficiently stiff that, upon rotating hub 40, torque may be transmitted to bent portion 117 (“J-tip”) of distal portion 116, thereby rotating bent portion 117 of distal portion 116. In examples, hub 40 may be rotated in a full 360 degree range, and bent portion 117 of distal portion 116 may similarly be rotated in a full 360 degree range of motion. Proximal portion 150 of shaft 100 and hub 40, as well as any unbent portions of distal portion 116, may rotate about line A (about a longitudinal axis of those portions). Distal tip 30 may move circumferentially about line A. Thus, rotation of hub 40/shaft 100 may enable manipulation of the guidewire advanced through lumen 112 (e.g., directional steering of the guidewire). In the relaxed/resting, “J-tip” configuration, there may be no sharp edges on distal portion 116, including distal tip 30, preventing damage to tissue as well as shearing of a guidewire extended through lumen 112.

With reference to FIG. 6, an accessory device 200 may be inserted into lumen 112 (e.g., via proximal opening 46 and lumen 42 of hub 40, shown in FIG. 1) and extended distally out of distal opening 114. In some examples, accessory device 200 may be a stylet, a needle, an awl, or another type of device. Accessory device 200 may be rigid and may have a sharpened distal tip 202. As shown in FIG. 6, when accessory device 200 is disposed within lumen 112, distal portion 116 may be straightened by accessory device 200. Distal tip 30 (which, as discussed above, which may be formed from jacket 130) may have a tapered shape that provides a smooth transition between more proximal portions of shaft 100 (e.g., a portion of distal portion 116 that is proximal of distal tip 30) to accessory device 200. Distal tip 30 may “hug” accessory device 200; in other words, a distalmost diameter of distal tip 30 may be only slightly larger than a diameter of accessory device 200. Upon removal of accessory device 200, distal portion 116 may resume a “J” shape (FIG. 2). Thus, when accessory device 200 extends from distal tip 30, a smooth taper may be formed between an outer diameter of accessory device 200, distal tip 30, and more proximal portions of shaft 100. A tapered shape of distal tip 30 may facilitate inserting shaft 100 into a target site after accessory device 200 punctures through tissue. As discussed below, accessory device 200 may puncture tissue, and then shaft 100 may be moved distally through the puncture, such that a portion of shaft 100 that is proximal of distal tip 30 extends into the tissue (e.g., to a target site). The smooth transition of distal tip 30 from accessory device 200 to more proximal portions of distal portion 116 may help to allow puncturing with minimal tissue tenting.

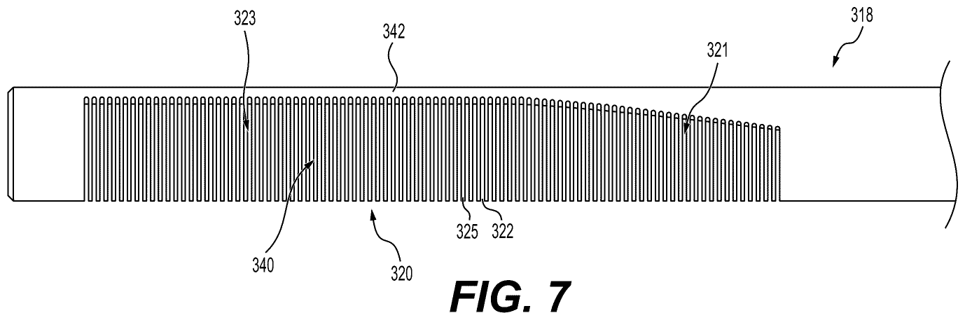

FIG. 7 depicts an alternative tube 318 that may be used in the alternative to tube 118. Unless otherwise stated, tube 318 may have any of the properties of tube 118. Tube 318 may have a slotted section 320. Slotted section 320 may include a single spine 342 running longitudinally along tube 318. A single array 340 of slots 322 may be formed. Slots 322 may extend along a circumference of tube 318, terminating at edges of spine 342. Slots 322 may form a plurality of rings 325 of tube 318, which are joined together at spine 342. As compared to tube 118, more material of tube 318 may be cut away by slots 322. This may provide greater flexibility of tube 318 as compared to tube 118 and may prevent potential interference between a sharp tip of accessory device 200 and cut portions of tube 318.

Similarly to array 140 of tube 118, array 340 may have a first portion 321 and a second portion 323. Like second portion 123, second portion 323 may have slots 322 of uniform length around a circumference of tube 118. Rings 325 of second portion 323 may similarly be uniform. Similar to first portion 121, first portion 321 may have slots 322 with a gradually tapering circumferential length. Circumferential lengths of slots 322 of first portion 321 may gradually decrease moving in a proximal direction (to the right in FIG. 7). As compared with slots 122 of tube 118, slots 322 may have a greater circumferential length, because tube 318 includes only one spine 342. Spine 342 may have an increasingly large circumferential length moving proximally along first portion 321. As compared with spines 141, spine 342 may have a larger circumferential length. As discussed above with respect to tube 118, the tapered shape of first portion 321 may help to provide strain relief.

Slots 122 and 322 of tubes 118 and 318, respectively, may additionally provide benefits to echogenic visualization of medical device 10. Medical device 10 may be utilized in procedures performed under endoscopic ultrasound (EUS) visualization, in which soundwaves may be emitted from a distal end of an endoscope (not shown). In general, rougher surfaces may have greater echogenic visualization properties because rougher surfaces diffuse reflection of the sound waves emitted by an ultrasound transducer. The scattered sound waves are more likely to return to the ultrasound transducer when a surface is rough, resulting in increased EUS visibility. Slots 122, 322 of tubes 118, 318 may result in diffuse reflection of sound waves that strike slots 122, 322, thereby producing enhanced visibility under EUS visualization.

As shown in FIG. 8, medical device 10 may be used in conjunction with or may have an electrosurgical component 400. As shown in FIG. 8, bent portion 117 may be bent such that a distalmost end of shaft 100 has an angle of greater than 90 degrees relative to a straight portion of shaft 100. Electrosurgical component 400 may include a sheath 410 and an electrosurgical tip 420. Shaft 100 may be disposed within a lumen 412 of sheath 410. Electrosurgical tip 420 may include a monopolar ring knife. An electrosurgical generator may be connected to a cautery connection in a handle (not shown) of medical device 10, and a wire may deliver energy to electrosurgical tip 420, which may act as a cutting surface. Electrosurgical tip 420 may have any of the features disclosed in U.S. patent application Ser. No. 16/941,913, published as U.S. Patent Application No. 2021/0030460, and incorporated herein in its entirety. Sheath 410 may be configured to be advanced/move over shaft 100 into a target site. For example, electrosurgical component 400 may be advanced over shaft 100 an a guidewire inserted through lumen 112, cutting and/or dilating tissue as it proceeds. Electrosurgical tip 420 may pass over the guidewire as it advances. Electrosurgical tip 420 may be energized in order to cut (vaporize) tissue during advancement into the target site.

Electrosurgical component 400 may have any features of any electrosurgical components known in the art. Jacket 130 (shown in FIG. 2) of medical device 10 may be configured to electrically insulate electrosurgical component 400 from tube 118 or 318. Jacket 130 may be thermally stable to withstand heat generated by electrosurgical component 400 as it cuts/vaporizes tissues. Electrosurgical component 400 may have any of the properties of electrosurgical sheaths, tips, and associated structures described in U.S. patent application Ser. No. 17/130,070, published as U.S. Patent Application Publication No. 2021/0205012, which is incorporated by reference herein in its entirety.

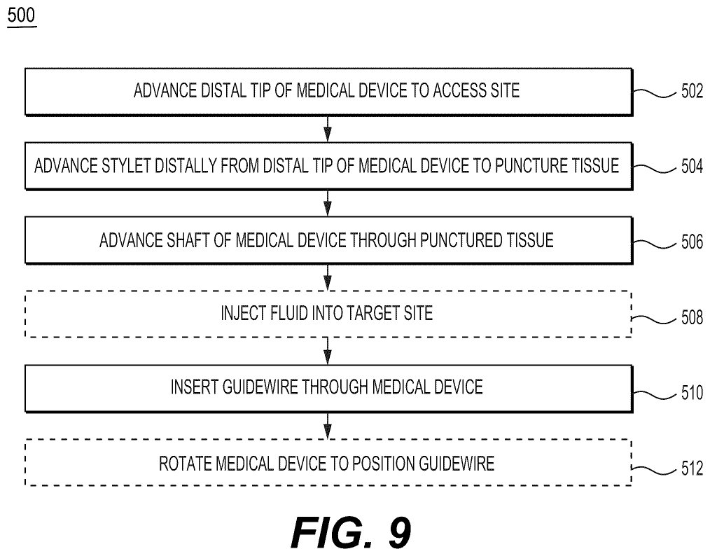

FIG. 9 depicts an exemplary method 500 for using medical device 10. Method 500 may be a portion of an EUS procedure used to access structures of a pancreatico-biliary of a subject such as, for example, when an endoscopic retrograde cholangiopancreatography (ERCP) procedure cannot be successfully performed. Method 500 may be performed under EUS guidance in order to direct a guidewire out of a papilla of Vater of a subject to perform a rendezvous procedure or in order to directly stent a dilated structure into a subject's duodenum or stomach. Access to a target site may be achieved via trans-gastric or trans-duodenal puncture into the target site.

In step 502 of method 500, medical device 10 may be advanced to an access site in a body of a subject. Distal tip 30 may be advanced to the access device and positioned where a user desires to puncture through tissue/gain access to a target site of the subject's body. In step 504, a stylet or other accessory device 200 may be advanced distally from distal tip 30 in order to puncture tissue of the subject's body. In some examples, step 502 may be performed with accessory device 200 already positioned in lumen 112 of shaft 100, either straightening distal portion 116 (as shown in FIG. 6) or with accessory device 200 proximal of bent portion 117, such that bent portion 117 has its relaxed configuration (as shown in FIG. 2). In other examples, step 502 may be performed without accessory device 200 positioned in lumen 112, and accessory device 200 may be inserted into lumen 112 in step 504. In examples, step 502 may include puncturing through a gastric wall or a wall of a duodenum (a trans-gastric or trans-duodenal puncture). For example, using the disclosed devices, a user may pass a stylet through medical device 10 in order to puncture into the target site and thereby access the target site. In step 506, shaft 100 of medical device 10 may be advanced through the tissue punctured in step 504. Step 506 may include inserting portions of shaft 100 that are proximal of distal tip 30 through the punctured tissue.

In an optional step 508, fluid may be injected into the target site via lumen 112 (e.g., via lumen 42 and opening 46 of hub 40). For example, contrast fluid may be injected into the target site. At any point in method 500, fluids may also be aspirated from the target site. For example, suction may be applied to lumen 112, and fluids may be aspirated through lumen 112. For example, step 508 may include confirming access to a target site by fluoroscopic contrast injection and/or aspiration of bile/pancreatic fluids through lumen 112. Additionally or alternatively, saline, sterile water, or another fluid may be injected via the medical device to help provide lubrication for a guidewire.

In step 510, a guidewire or other type of accessory device may be inserted into lumen 112 (e.g., via lumen 42 and opening 46 of hub 40). In examples, accessory device 200 may be removed prior to step 510. In aspects, accessory device may be removed prior to step 508 or following step 508. After accessing the target site, a guidewire may be passed through the medical device to, for example, advance through the papilla for a rendezvous procedure or to secure access for trans-mural stenting. The guidewire may extend through distal opening 114. Optionally, in step 512, hub 40 or another element of device 10 may be rotated in order to change an orientation of distal portion 116 (which may have a “J-tip” shape) in order to direct the guidewire or other accessory device to a desired location. Steps 510 and/or 512 may be utilized to advance a guidewire through the papilla of a subject for a rendezvous procedure or to help secure access for trans-mural stenting. Because medical device 10 may have an atraumatic tip, manipulation of the guidewire may be achieved without damaging nearby tissue or shearing the guidewire. Thereafter, a desired procedure may be performed using the guidewire. For example, electrosurgical tip 420 may be activated and/or advanced over the guidewire. In examples, electrosurgical tip 420 may be deactivated, advanced, and reactivated, or may be advanced while electrosurgical tip 420 is activated.

It will be apparent to those skilled in the art that various modifications and variations can be made to the disclosed device without departing from the scope of the disclosure. Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A medical device comprising:
a tube including:
a proximal portion; and
a distal portion having a slotted section with a plurality of slots formed therein, wherein a proximalmost slot of the plurality of slots has a first length in a circumferential direction about the tube, wherein a distalmost slot of the plurality of slots has a second length in the circumferential direction about the tube, and wherein the first length is smaller than the second length; and
a hub coupled to a proximal portion of the tube, wherein rotation of the hub is configured to rotate the proximal portion of the tube about a longitudinal axis of the proximal portion of the tube.

2. The medical device of claim 1, wherein each slot of the plurality of slots extends approximately perpendicularly to a longitudinal axis the tube.

3. The medical device of claim 1, wherein the slotted section includes a longitudinally extending unslotted spine.

4. The medical device of claim 3, wherein the spine is a first spine, and wherein the slotted section includes a second longitudinally extending unslotted spine.

5. The medical device of claim 4, wherein each slot of the plurality of slots extends between the first spine and the second spine.

6. The medical device of claim 1, wherein the tube includes an array of slots having a first, proximal portion of slots and a second, distal portion.

7. The medical device of claim 6, wherein each slot of the plurality of slots of the second portion has approximately a same circumferential length.

8. The medical device of claim 7, wherein lengths of the plurality of slots in the first portion taper from a first circumferential length at a proximal end of the first portion to a second circumferential length at a distal end of the first portion, and wherein the second circumferential length is greater than the first circumferential length.

9. The medical device of claim 1, wherein, in a relaxed state, the distal portion includes a curved portion having a curved central longitudinal axis.

10. The medical device of claim 9, wherein, in the relaxed state, the tube has an approximately “J” shape.

11. The medical device of claim 1, further comprising a jacket covering at least a portion of the tube.

12. The medical device of claim 11, wherein the jacket extends distally beyond a distalmost end of the tube such that the jacket forms a distal tip of the medical device.

13. The medical device of claim 12, wherein the distal tip tapers radially inward toward a distal end of the distal tip.

14. The medical device of claim 1, further comprising an electrosurgical component that is configured to advance over the tube.

15. The medical device of claim 1, wherein the distal portion further includes an unslotted section distal to the slotted section.

16. A medical device comprising:
a tube including:
a proximal portion; and
a distal portion having a slotted section, wherein the slotted section includes:
a longitudinally extending unslotted spine; and
a plurality of slots extending from the unslotted spine;
wherein, in a relaxed state, the distal portion includes a curved portion having a curved central longitudinal axis; and
a hub coupled to a proximal portion of the tube.

17. The medical device of claim 16, wherein the spine is a first spine, and wherein the slotted section includes a second longitudinally extending unslotted spine.

18. The medical device of claim 17, wherein each slot of the plurality of slots extends between the first spine and the second spine.

19. A medical device comprising:
a tube including:
a proximal portion; and
a distal portion having an array of slots having a first, proximal portion of slots and a second, distal portion of slots, wherein each slot of the second portion has approximately a same circumferential length, wherein lengths of the slots in the first portion taper from a first circumferential length at a proximal end of the first portion to a second circumferential length at a distal end of the first portion, and wherein the second circumferential length is greater than the first circumferential length; and
a hub coupled to a proximal portion of the tube.

20. The medical device of claim 19, wherein, in a relaxed state, the distal portion includes a curved portion having a curved central longitudinal axis.
